(19) United States
(12) Patent Application Publication
(10) Pub. No.: US 2008/0194686 A1
(43) Pub. Date: Aug. 14, 2008

(54) INDUCTION OF BETAINE-HOMOCYSTEINE S-METHYLTRANSFERASE TO ELEVATE APOLIPOPROTEIN B AND VERY-LOW-DENSITY LIPOPROTEIN

(75) Inventors: Charles E. Sparks, Pittsford, NY (US); Janet D. Sparks, Pittsford, NY (US); Mark P. Sowden, Penfield, NY (US)

Correspondence Address: McDERMOTT WILL & EMERY LLP, Suite 500, 18191 Von Karman Avenue, Irvine, CA, 92612, US

(73) Assignee: UNIVERSITY OF ROCHESTER, Rochester, NY (US)

(21) Appl. No.: 11/963,722

(22) Filed: Dec. 21, 2007

Related U.S. Application Data
(60) Provisional application No. 60/878,528, filed on Jan. 3, 2007.

(51) Int. Cl. A61K 31/195; A61P 1/16

(52) U.S. Cl. 514/561

(57) ABSTRACT

Methods for use in mobilizing triacylglycerol (TAG) in order to elevate apoliprotein B and Very-Low-Density Lipoprotein (VLDL) are described. In some embodiments, the method involves providing betaine in an amount of about 3 gm/kg of body weight per day in order to mobilize TAG. In some embodiments, serum VLDL is elevated as a result of mobilizing hepatic TAG. In some embodiments, the method includes restricting methionine intake to less than about 1 gm/kg of body weight per day. In some embodiments, the method includes feeding L-cysteine at a level of at least about 3 gm/kg of body weight per day. In some embodiments, the method includes feeding choline bitartrate at a level of about 1.25 gm/kg of body weight per day. Mobilizing hepatic TAG is effective to reduce hepatic steatosis.

### RELATED APPLICATIONS

This application claims priority to U.S. Provisional Application No. 60/878,528, filed Jan. 3, 2007, the contents of which are herein incorporated by reference in their entirety.

### STATEMENT REGARDING FEDERALLY FUNDED RESEARCH

The U.S. Government has a paid-up license in this invention and the right in limited circumstances to require the patent owner to license others on reasonable terms as provided for by the terms of grant numbers HL-065398 and DK-043739, awarded by the National Institutes of Health.

### SUMMARY OF THE INVENTION

In some embodiments, there is provided a method, for mobilizing triacylglycerol in a mammal, the method comprising: feeding the mammal a diet that includes betaine and restricts methionine. In some embodiments, the triacylglycerol that is mobilized is hepatic triacylglycerol. In some embodiments, the betaine is fed at or above a level of approximately 3 grams per kilogram of body weight per day. In some embodiments, the method further comprises elevating a serum VLDL level in the mammal as a result of the mobilizing of hepatic triacylglycerol. In some embodiments, the method further comprises elevating a serum apolipoprotein B level in the mammal as a result of the mobilizing of hepatic triacylglycerol. In some embodiments, the methionine is fed at or below a level of approximately 1 gram per kilogram of body weight per day. In some embodiments, the method further comprises feeding L-cysteine at or above a level of approximately 3 grams per kilogram of body weight per day. In some embodiments, the method further comprises feeding choline bitartrate at or above a level of approximately 1.25 grams per kilogram of body weight per day. In some embodiments, the method further comprises reducing hepatic steatosis in the mammal as a result of the mobilizing of hepatic triacylglycerol.

### DETAILED DESCRIPTION OF EXEMPLARY EMBODIMENTS

Please see the appended publication: Janet D. Sparks, Heidi L. Collins, Doru V. Chirieac, Joanne Cianci, Jenny Jokinen, Mark P. Sowden, Chad A. Galloway & Charles E. Sparks, Hepatic very-low-density lipoprotein and apolipoprotein B production are increased following in vivo induction of betaine-homocysteine S-methyltransferase, Biochem J. (2006) 395, 363-371.

What is claimed is:

1. A method, for mobilizing triacylglycerol in a mammal, the method comprising:
feeding the mammal a diet that includes betaine and restricts methionine.

2. The method of claim 1, wherein the triacylglycerol that is mobilized is hepatic triacylglycerol.

3. The method of claim 1, wherein the betaine is fed at or above a level of approximately 3 grams per kilogram of body weight per day.

4. The method of claim 1, further comprising elevating a serum VLDL level in the mammal as a result of the mobilizing of hepatic triacylglycerol.

5. The method of claim 1, further comprising elevating a serum apolipoprotein B level in the mammal as a result of the mobilizing of hepatic triacylglycerol.

6. The method of claim 1, wherein the methionine is fed at or below a level of approximately 1 gram per kilogram of body weight per day.

7. The method of claim 1, further comprising feeding L-cysteine at or above a level of approximately 3 grams per kilogram of body weight per day.

8. The method of claim 1, further comprising feeding choline bitartrate at or above a level of approximately 1.25 grams per kilogram of body weight per day.

9. The method of claim 1, further comprising reducing hepatic steatosis in the mammal as a result of the mobilizing of hepatic triacylglycerol.
